(19) United States
(12) Patent Application Publication
(10) Pub. No.: US 2026/0235596 A1
(43) Pub. Date: Aug. 13, 2026

(54) POLYMER COATED MAGENTIC BEADS SERVING AS RAMAN REPORTER FOR SIMULTANEOUS TARGET BINDING AND IDENTIFICATION

(71) Applicant: Massachusetts Institute of Technology, Cambridge, MA (US)

(72) Inventors: Loza TADESSE, Cambridge, MA (US); Marissa Rose MCDONALD, Cambridge, MA (US); Jongwan LEE, Cambridge, MA (US); Nikiwe MHLANGA, Cambridge, MA (US)

(21) Appl. No.: 19/471,814

(22) PCT Filed: May 9, 2024
(86) PCT No.: PCT/US2024/028577

§ 371 Date: Oct. 2, 2025

Related U.S. Application Data
(60) Provisional application No. 63/501,380, filed on May 10, 2023.

(51) Int. Cl. G01N 33/543; G01N 21/65

(52) U.S. Cl. CPC G01N 33/54326; G01N 21/65

(57) ABSTRACT

Methods of detection are provided, comprising (i) obtaining a. sample, wherein the sample comprises target selected from biological cells and viruses; (ii) incubating the sample with polymer-coated magnetic beads to produce bead-target complexes; (iii) analyzing the bead-target complexes by Raman spectroscopy to produce a spectrograph; and (iv) detecting the presence of the bead-target complexes by analyzing the spectrograph.

### BACKGROUND

Immunomagnetic separation is a technique that utilizes antibody-coated superparamagnetic beads that target antigens on cell surfaces to capture and concentrate cells. Since their invention more than 4 decades ago^{1,2}, polymer-coated magnetic beads, such as Dynabeads™ (ThermoFisher Scientific™, Inc.), have been incorporated into routine biological experiments and even notable clinical trials such as in the isolation of CD34+ bone marrow-derived stem cells and CD3+/CD28+ T-cells in novel adoptive immunotherapy.³ Dynabeads™ are the most frequently cited tool for immunomagnetic separation and result in high purity (95-100%) and viability (60-95%) of captured cells.^{4,5} Particularly, their extremely versatile target-specific antibody-coupled surface enables the capture and magnetic separation of intact target cells from heterogeneous liquid samples such as blood and wastewater, eliminating the need for column separation or centrifugation techniques. Their superparamagnetic iron oxide core and tunable surface functionalities enable them to be magnetically activated in the presence of an external magnetic field, allowing for dispersion in solution when the magnetic field is no longer applied and the rapid and gentle isolation of target cells when needed. Moreover, the polystyrene coating shields targets from the cytotoxic iron oxide core, making the beads biocompatible. Although cell sorting methods like fluorescence-activated cell sorting (FACS) are extremely effective, they require large concentrations of cells, tedious sample preparation steps, expensive cell-specific labels, and highly skilled personnel for use and maintenance, making them challenging for high throughput and field deployable applications.⁶ Despite the immense benefits of polymer-coated magnetic beads, detection of captured targets remains a challenge. Current approaches still rely on traditional, time-consuming culturing or involved and tedious, molecular-based techniques such as polymerase chain reaction (PCR), enzyme immunoassays (EIA), and matrix-assisted laser desorption/ionization time of flight mass spectroscopy (MALDI-TOF MS) which suffer from downsides similar to FACS.⁷⁻⁹ However, with emerging methods to separate free polymer-coated magnetic beads from polymer-coated magnetic beads bound to targets, specific detection of polymer-coated magnetic beads bound to targets in lieu of detecting the target is becoming possible.¹⁰ Thus, a scalable and simple detection scheme leveraging this approach is needed to exploit the full versatility of polymer-coated magnetic beads for widespread use.

Raman spectroscopy is an emerging biosensing approach that generates molecular fingerprints of targets by using the inelastic scattering of light from samples.¹¹ In addition to scientific grade tabletop versions, cost effective, portable Raman systems are improving its accessibility for field applications. Thus, a polymer-coated magnetic beads-based Raman spectroscopy assay presents an opportunity for rapid identification of magnetically concentrated targets. Studies in this area thus far, however, have focused on recording the signature of target cells or biomolecules, which typically generate weak Raman signals, and therefore relied on engineering complex dual substrates with a magnetic core and surrounding plasmonic metal nanoparticles such as gold and silver. Engineering these unique substrates adds complex chemical synthesis schemes that can be challenging for widespread translation and are vulnerable to irreproducible enhancements of target signals, artifacts from antibodies on beads and other contaminants in the sample, due to off-target enhancement by metal nanoparticles. In contrast, similar to fluorescent probes, Raman probes can serve as a reporter of bound targets providing a strong Raman signature of their own fingerprint, which can indirectly signal the presence of the target. However, adding such probes to substrates also suffers from similar drawbacks as plasmonic substrate engineering.

### SUMMARY

This Summary introduces a selection of concepts in simplified form that are described further below in the Detailed Description. This Summary neither identifies key or essential features, nor limits the scope, of the claimed subject matter.

A first aspect of the disclosure here is a method of detection, wherein the method comprises
obtaining a sample, wherein the sample comprises target selected from biological cells and viruses;
incubating the sample with polymer-coated magnetic beads that comprise an affinity agent for targeton a bead surface, to produce bead-target complexes;
analyzing the bead-target complexes by Raman spectroscopy to produce a spectrograph; and
detecting the presence of the bead-target complexes by analyzing the spectrograph.

As disclosed in the examples that follow, the inventors have demonstrated the use of polymer-coated magnetic beads, exemplified by Dynabeads™, as strong Raman reporters themselves without additional chemical labeling for simultaneous isolation and indirect detection of targets, both in dried and liquid sample preparation formats. Specifically, the inventors utilizes polymer-coated magnetic beads coated with anti-Salmonella antibodies for indirect detection of Salmonella enterica, the leading cause of hospitalizations and death due to foodborne illness, demonstrating the ability to both magnetically isolate cells in the sample and provide strong Raman signal towards a rapid, sensitive, and specific approach for bacterial detection. This technique can be adapted to probe for any biological cell or viral target using polymer-coated magnetic beads comprising an affinity agent for cells or viruses of interest, in any assay format, including but not limited to lateral flow assay or vertical separation formats.

Any sample containing or suspected of containing biological cells or viruses, may be used in the methods of the disclosure. The biological cells may be any cells of interest to a user. The cells may comprise prokaryotic or eukaryotic cells. In non-limiting embodiments, the cells may comprise mammalian cells or human cells. In other non-limiting embodiments, the cells may comprise, tumor cells, cancer stem cells, hematopoietic stem and progenitor cells, mesenchymal stem and progenitor cells, adipose-derived stem and progenitor cells, endothelial progenitor cells, white blood cells, granulocytes, mononuclear cells, lymphocytes, monocytes, T-cells, B-cells, NK cells, CAR-T cells, genetically modified cells, bacterial cells, fungi. Similarly, the virus may be any virus of interest to an end user.

In one embodiment, the sample is a biological sample obtained from a subject, including but not limited to blood, saliva, urine, stool, sweat, vaginal secretion, semen, cheek swab, etc. In other non-limiting embodiments, the sample may be a food sample, a water sample (water source; wastewater; etc.), a solid waste sample, an air sample, etc.

The magnetic beads may comprise any suitable magnetic material in or surrounding the bead core, including but not limited to iron oxide, nickel oxide, cobalt oxide, etc. In one embodiment, the magnetic material comprises iron oxide. In some embodiments, the magnetic material is superparamagnetic, including but not limited to superparamagnetic iron oxide.

The polymer coating may comprise any polymer suitable for an intended purpose of the magnetic beads. In various embodiments, the polymer may comprise polystyrene, polyacrylamide, triblock polymer polyisopropene-block-poly(2-cinnamnoylethyl methacrylate)-block-poly(tert-butyl acrylate), dextran, alginate, poly(vinyl alcohol) (PVA), copolymers of acetoacetoxyethyl methacrylate and N-vinylcaprolactam, and copolymers of N-isopropyl acrylamide (NIPA) and glycidyl methacrylate. In one embodiment, the polymer-coated magnetic beads comprise polystyrene-coated beads.

In various embodiments, the polymer-coated magnetic beads are between 1 μm and 10 μm in diameter, or between 1 μm and 5 μm in diameter, are non-porous, and comprise superparamagnetic iron oxide coated with polystyrene. In some such embodiments, the polymer-coated magnetic beads are Fe₂O₃ nanoparticles with sizes ranging from 6-12 nm that occasionally form 20 nm clusters. In one embodiment, the polymer-coated magnetic beads may be purchased commercially under the trade name Dynabeads™ (ThermoFisher Scientific, Inc.).

The polymer-coated magnetic beads comprise an affinity agent for the target on a bead surface (i.e.: the affinity agent binds to the target to permit formation of the bead-target complex). The affinity agent may be any moiety that binds to the microbe. In one embodiment, the affinity agent is an antibody that binds to the target. As shown in the examples that follow, the inventors utilized polymer-coated magnetic beads coated with anti-Salmonella antibodies for indirect detection of Salmonella enterica. Those of skill in the art will understand, based on the teachings herein, that any other antibody for any other target may be used in the methods of the disclosure. The affinity agent/antibody may be coated on the bead surface using standard techniques in the art.

The incubation occurs under conditions suitable for binding of the affinity agent and the target. Exemplary such conditions are as described in the examples that follow. It is well within the level of skill in the art, based on the teachings herein, to determine appropriate assay conditions for a particular use.

The incubating results in formation of a bead-target complex, and the method further comprises analyzing the bead-target complexes by Raman spectroscopy to produce a spectrograph. Raman spectroscopy employs inelastic scattering of photons, known as Raman scattering. Typically, a laser is used, and light from the laser interacts with molecular vibrations, phonons or other excitations in the system, resulting in the energy of the laser photons being shifted up or down. The shift in energy gives information about the vibrational modes in the system. Exemplary conditions for carrying out Raman spectroscopy and detection of the bead-target complex are provided in the examples herein.

In one embodiment, analyzing the bead-target complex by Raman spectroscopy measures polystyrene of the polystyrene-coated bead. In one embodiment, analyzing the bead-target complex measures Raman intensity at 700 to 1700 cm⁻¹.

In one embodiment of the disclosed method, the bead-target complex has a different density than the polymer-coated magnetic bead.

In one embodiment, the disclosed method further comprises separating the bead-target complex from the polymer-coated magnetic bead. In one embodiment of the disclosed method, a density gradient is used in separating the bead-target complex from the polymer-coated magnetic bead. In one embodiment, the density gradient comprises two layers, wherein the density of the two layers differs. In one embodiment, the density gradient comprises two layers, wherein the density of the two layers differs. In one embodiment, the density gradient comprises a continuous density gradient.

In one embodiment of the disclosed method, the analyzing the bead-target complex by Raman spectroscopy measures only the bead-target complex and not polymer-coated magnetic beads free of targets. In one embodiment, separating the bead-target complex is performed based on the density of the bead-target complex. In one embodiment, separating the bead-target complex is performed based on the magnetic properties of the polymer-coated magnetic bead.

A second aspect of the disclosure herein is a method of enriching, the method comprising
obtaining a sample, wherein the sample comprises target selected from biological cells and viruses;
incubating the sample with polymer-coated magnetic beads that comprise an affinity agent for the target on a bead surface, to produce bead-target complexes; and
separating the bead-target complexes from the polymer-coated magnetic beads based on the density of the bead-target complex;
wherein the separating the bead-target complex enriches a relative concentration of the bead-target complexes.

All embodiments and combinations of the first aspect of the disclosure may be used in this second aspect. In one embodiment, separating the bead-target complex is performed based on the magnetic properties of the polymer-coated magnetic beads.

In one embodiment, the affinity agent comprises an antibody, wherein the antibody binds to target.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

FIG. 1. Overview of Raman setup and Dynabeads™ employed. (A) Liquid well imaging setup with S. enterica and Dynabeads™ suspended in DIW being pulled down to the imaging surface with a magnet prior to Raman interrogation with a 785 nm laser. Schematic created with Biorender.com (B) transmission electron micrograph (TEM) of (left) rod-shaped S. enterica bacteria target, (middle) 2.5 um Dynabeads™, and (right) S. enterica-bound Dynabeads™, showing multiple cells binding to each bead and across beads. (C) UV-Vis absorption spectra of Dynabeads™, S. enterica, and Dynabeads™-bound S. enterica, showing broad and large absorbance of Dynabeads™ primarily due to the iron oxide core as confirmed by later analyses.

FIG. 2. Raman interrogation of dried samples from single point detection. (A) Brightfield images of S. enterica, Dynabeads™ (single and clustered), and S. enterica-bound Dynabeads™ (single and clustered) showing areas where the incident laser was focused for Raman measurements. (B) Corresponding Raman spectra showing clear Raman signature of beads with major peaks at 1000, 1350, and 1600 cm⁻¹, which are signatures of polystyrene and antibody coating on bead surface as shown in FIGS. 11 and 12. This unique Raman signature from Dynabeads™ reporters is maintained upon conjugation with cells, demonstrating the signal is coming from the beads and highlighting their strong Raman reporter capability after target capture. (C) Analysis of Raman intensities from Dynabeads™ samples with respect to Raman signature from bacteria alone. Intensities from selected wavenumbers (1000, 1350, and 1600 cm⁻¹) corresponding to the signature peaks of Dynabeads™ from each sample were divided with the respective intensities of S. enterica to calculate the intensity difference. All analysis was performed from data collected at four different sample locations, as shown in FIG. 10. Overall, the highest intensity is recorded at the 1000 cm⁻¹ peak; single S. enterica-bound Dynabeads™ have lower signal intensities than single unbound Dynabeads™, but clusters of bound beads have mostly comparable intensities at the signature peaks compared to clusters of unbound Dynabeads™.

FIG. 3. Raman mapping of dried samples at 0.5 s acquisition for high throughput signal detection. (A) Brightfield images and intensity maps of S. enterica, Dynabeads™ (single or clustered), and S. enterica-bound Dynabeads™ (single or clustered) with dotted circles showing regions where the incident laser was focused and whose intensity maps and spectra are shown below. All scale bars are 10 μm. (B) Raman spectra from S. enterica, single Dynabeads™, and single S. enterica-bound Dynabeads™ collected with a single shot 0.5 second acquisitions with 7 mW laser power across a 30×30 μm area. Signature peaks of Dynabeads™ at 1000, 1350, and 1600 cm⁻¹ are noted. Spectra shows matching signatures in both bound and unbound beads, confirming that the signal source is the Dynabeads™. (C) Raman spectra from S. enterica, clustered Dynabeads™, and clustered S. enterica-bound Dynabeads™ with higher intensities observed for bead-only clusters.

FIG. 4. Raman interrogation in liquid samples with 0.5 s acquisition at 75 mW laser power. (A) Brightfield images and intensity maps of S. enterica, Dynabeads™ (single and clustered), and S. enterica-bound Dynabeads™ (single and clustered) showing dislocation of cell or bead only samples upon laser exposure. In contrast, cell-bound beads (single and clustered) stay in place after laser exposure. (B-C) Raman spectra of S. enterica and single or clustered Dynabeads™ only and S. enterica-bound Dynabeads™. Both single and clustered Dynabeads™ only samples show inhibited detection due to dislocation of Dynabeads™ upon laser exposure from heat-induced convective fluid flow. S. enterica-bound Dynabeads™, in both single and clustered format, remain in place at the bottom of the well upon laser exposure and show similar spectral features as dried samples, with larger intensity from bacteria-bound clusters.

FIG. 5. Characterization of Dynabeads™ anti-Salmonella. (A) scanning electron microscopy (SEM) and TEM of Dynabeads™ showing (left) uniform size distribution, (middle) porous surface morphology, and (right) widths of iron oxide core (~2.44 μm) and polystyrene shell (~0.15 μm). (B) EDX spectrum of Dynabeads™, showing significant contributions from iron and oxygen. Inset shows EDX mapping image of two Dynabeads™ with corresponding regions of iron (middle) and oxygen (right). Oxygen distribution is uniform throughout the core, but iron distribution is lopsided. A major carbon peak and minor copper and sulfur peaks are observed which are from the polymer coating, TEM grid and antibody coating respectively.

FIG. 6. Fluorescence microscopy of S. enterica (left), polymer-coated beads (middle), and S. enterica-bound beads (right) showing size, shape, and tight binding interaction. All scale bars are 5 μm.

FIG. 7. UV-Vis spectra of two batches of polymer-coated beads and S. enterica-bound beads showing consistent absorbance trends across batches. Decreases in absorbance from Batch 2 could be due to the degradation of the beads as they decay past their shelf life.

FIG. 8. Clustering of beads in dried and liquid samples before and after conjugation to S. enterica. All scale bars are 10 μm.

FIG. 9. Preparation of samples for Raman interrogation. (A) Optical images of dried polymer-coated beads with 0, 1, and 2 washing cycles. Most residues in the stock buffer (sodium azide and BSA) can be removed after two cycles of washing. (B) Beads and S. enterica samples were prepared at a fixed concentration of 10⁷ beads/mL and 5×10⁶ cells/mL respectively. For conjugation, a ratio of 1:0.5 beads to S. enterica was used.

FIG. 10. Raman spectra from dried samples. (A) Data from four different sample locations were collected and analyzed (scale bar=5 μm). (B) S. enterica (top), single-and clustered Dynabeads™ (middle), and single-and clustered S. enterica-bound Dynabeads™. Signal from clustered beads is overall larger than that of single beads in both conditions.

FIG. 11. Raman spectra from polystyrene (PS). (A) Brightfield images of PS with different molecular weights (M_{w}): 35,000 and 350,000 g/mol. Images show where the incident laser was focused for Raman interrogation. (B) Raman spectra of PS of different M_{W}. Prominent peaks at 1000 and 1600 match observation in Dynabeads™-containing samples, indicating large contributions of polystyrene to Raman spectra of Dynabeads™.

FIG. 12. Raman spectra of anti-Salmonella antibodies with 1 min acquisition at 100 mW. Major peaks appear at 1004, 1243, 1452, and 1674 cm-1. While some peaks may be present in the signature of the beads, they appear with much less intensity contribution than those of polystyrene (FIG. 11), suggesting that the Raman signal of surface anti-Salmonella antibodies is contributing very little if at all to the Raman signal of the anti-Salmonella beads. This is as expected given that antibodies typically present low spontaneous Raman signals. This is further corroborated by findings in FIG. 13.

FIG. 13. Raman map from dried samples. (A) Data from three different locations were collected and analyzed (scale bar=10 μm). Highly concentrated pellets were selected as S. enterica region. Locations with visible S. enterica and Dynabeads™ in the form of single and clustered formats are selected as S. enterica-bound Dynabeads™ region. (B) Intensity maps after combining intensities at selected wavenumbers; 1,000, 1,350, and 1,600 cm⁻¹. At this acquisition parameter no detectable signature is observed from S. enterica only sample, mainly background intensity is detected. (C) Raman spectra from S. enterica do not show specific signature peaks under the 0.5 sec exposure 7.5 mW-power laser acquisition parameters. In contrast, Dynabeads™ alone and S. enterica-bound Dynabeads™ show strong signature peaks from locations circled in dotted lines in the bright field images in A with notable peaks at 1000, 1350, and 1600 cm⁻¹. (D) Intensity difference factors as compared to the bacteria only baseline shows a similar trend as the single point measurement in FIG. 2, with overall higher intensity recorded from clustered Dynabeads™.

FIG. 14. Dislocation of Dynabeads™ and S. enterica-bound Dynabeads™ before/after exposure to 75 mW-powered laser (L=785 nm) for 0.5 sec. (A) Each sample was observed using a brightfield microscope, top showing before and bottom after exposure to laser. (B) Classification of samples in FOV, as single (N≤2) and clustered (N≥3) forms, showing distinct differences in Dynabeads™ and S. enterica-bound Dynabeads™; all of Dynabeads™ were dislocated, but none to few S. enterica-bound Dynabeads™ did. (C) Quantitative analysis on the composition of the number of single (N_{S}) and clustered (Nc) samples. For Dynabeads™, all samples were entirely dislocated. Two single Dynabeads™ were observed in FOV for single bead measurement before laser exposure, but they were dislocated and replaced by a cluster after exposure. In the case of single bead measurement, pre-exposure one single Dynabeads™ (1.2% composition) and cluster of 55 Dynabeads™ (98.2%) were observed, post laser exposure all of Dynabeads™ were dislocated from FOV, resulting in 0% of each composition. For single S. enterica-bound Dynabeads™, three single samples (100% in the composition of the singles) were observed, and they retained their position post laser exposure as shown in FIG. 2. For clustered S. enterica-bound Dynabeads™, two single (5.7% in composition)- and cluster of 33 (94.3%) were observed pre-exposure, and one single (3.8%)- and cluster 0f 25 (96.2%) were observed post exposure. (D) Generation of bubbles inside clustered S. enterica-bound Dynabeads™ induced by the laser exposure.

FIG. 15. Effects of laser exposure on sample dislocation in liquid. (A) Brightfield images of each sample before- and after laser exposure during Raman interrogation. Only S. enterica-bound Dynabeads™ remain in place post-exposure. (B) Raman spectra from each sample. The spectra from S. enterica-bound Dynabeads™ showed the highest intensities compared to other samples.

FIG. 16. EDX images of Dynabeads™ (left) with corresponding regions of iron (middle) and oxygen (right). Oxygen distribution is uniform throughout the core, but iron distribution favors one side of the bead. All scale bars are 2 m.

FIG. 17. Focused ion beam (FIB) analysis of Dynabeads™. (A) Cross sectional image of 4 beads compared to 3 uncut beads also in the field of view. (B) Close up image of cross-sectional view of Dynabeads™ exposes amorphous structure with no specific physical pattern of iron oxide core particles except for faint white dots.

FIG. 18. Raman interrogation of buffer solutions. (A) Brightfield images and Raman spectra from dried cell culture media (TSB). (B) Brightfield images and Raman spectra from Dynabeads™ stock buffer (PBS, BSA, sodium azide). Both A and B demonstrate that the observed Raman signal of Dynabeads™ is not coming from residual buffer solution.

### DETAILED DESCRIPTION

Reference numbers in superscripts herein refer to the corresponding literature listed in the attached bibliography, which forms a part of this Specification, and the literature is incorporated by reference herein.

What is disclosed herein is the use of polymer-coated magnetic beads, exemplified by magnetic Dynabeads™ as strong Raman reporters themselves without additional chemical labeling for simultaneous isolation and indirect detection of targets. We demonstrate detection of the Raman reporter signature of Dynabeads™ both in dried and liquid sample preparation formats. Specifically, we utilize Dynabeads™ coated with anti-Salmonella antibodies for indirect detection of Salmonella enterica, the leading cause of hospitalizations and death due to foodborne illness.^{15,16} We note the dual function of Dynabeads™ coated with species-specific antibodies to both magnetically isolate cells in the sample and provide strong Raman signal towards a rapid, sensitive, and specific approach for bacterial detection. This technique can be adapted to probe for any target using target-specific Dynabeads™ in a lateral flow assay or vertical separation formats that can isolate target bound-Dynabeads™ from free Dynabeads™. Here even single to few target bound-Dynabeads™ can present detectable Raman signature providing indirect confirmation of presence of a handful of target cells without the need for further culturing or staining steps.

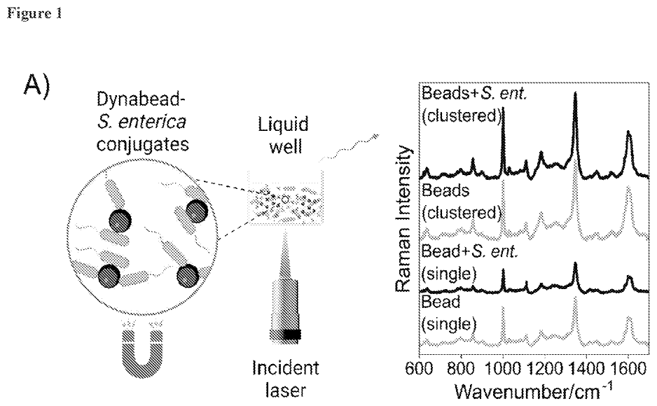

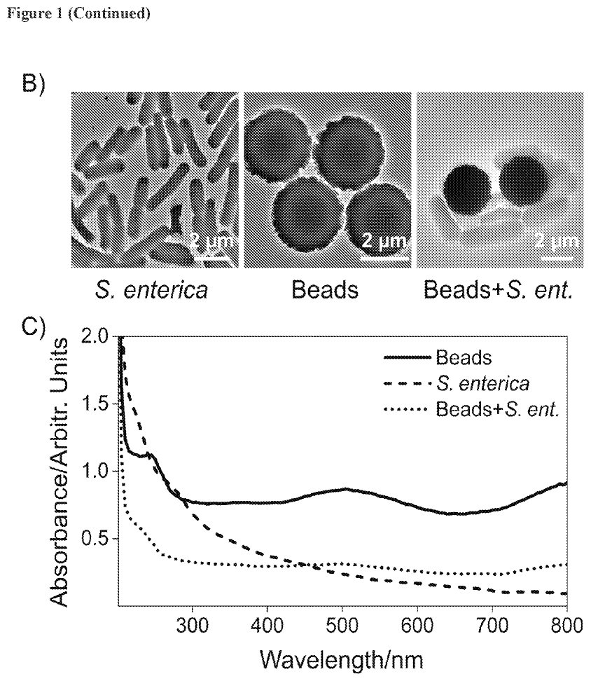

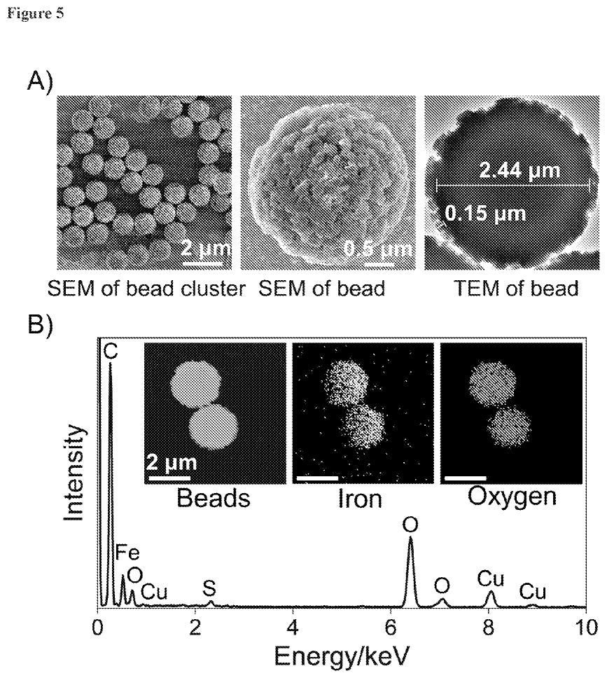

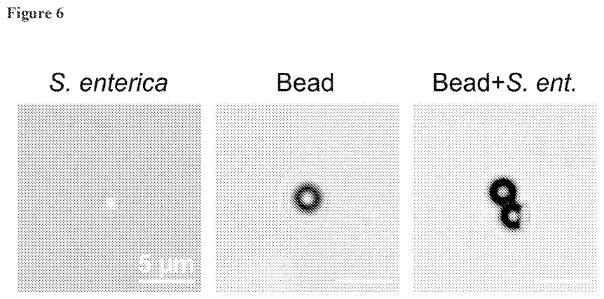

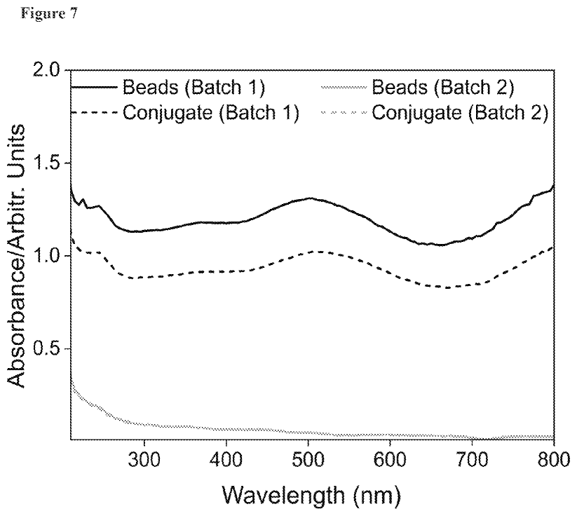

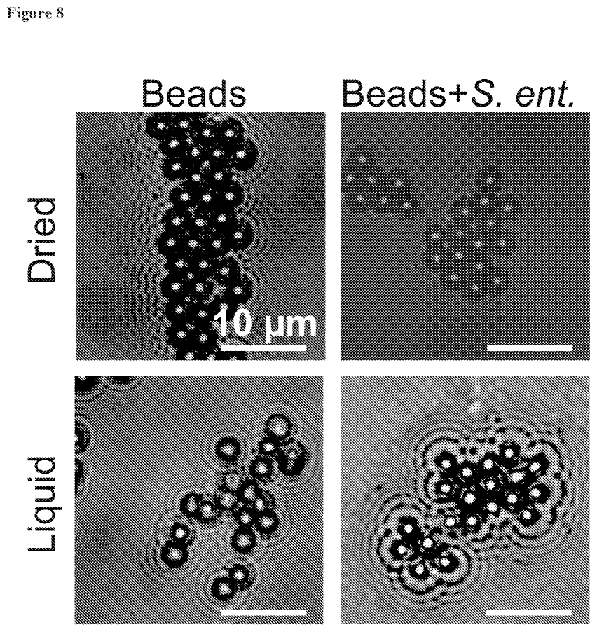

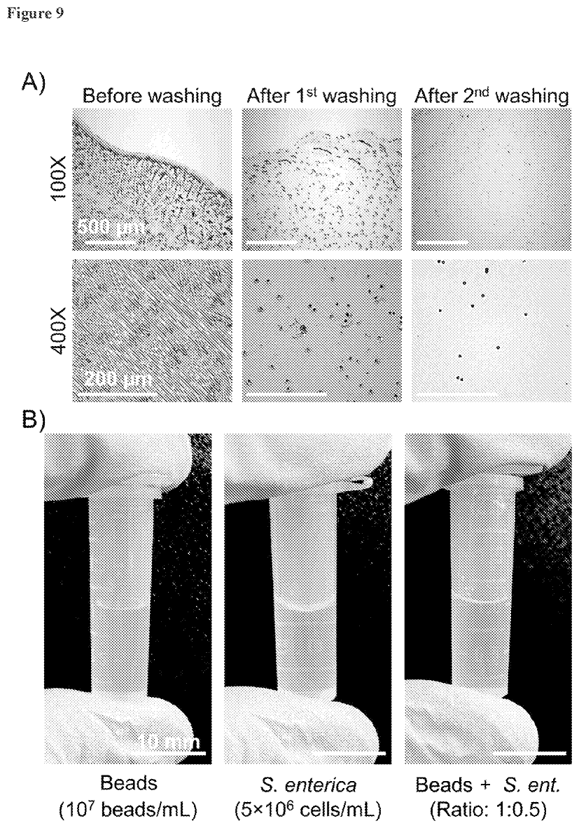

Our experimental setup for detection of Salmonella from liquid samples is summarized in FIG. 1. Conjugates of Dynabeads™ anti-Salmonella and S. enterica were formed and suspended in a liquid well of deionized water (DIW) with optically transparent quartz substrate. Here, we aim to demonstrate the Raman reporter nature of Dynabeads™ in target sample types and measurement conditions, however the implementation of this Raman based detection scheme is adoptable to the respective target-bound bead separation assay of choice. Taking advantage of the superparamagnetic property of Dynabeads™, the conjugates were concentrated down to the imaging surface using a magnet immediately before interrogation with a 785 nm incident laser focused at the bottom of the well as shown in FIG. 1A. This magnetic concentration step semi-fixed bead-bound cells in the field of view rather than having them freely float in the liquid. The target S. enterica, as shown in its TEM, is a rod-shaped bacteria with a 2-3 m length and 0.7-1 m width (FIG. 1). Dynabeads™ are ~2.5 μm diameter spherical particles with a polystyrene polymer shell coupled to species-specific antibodies. As shown on FIG. 1B and FIG. 6, conjugation of Dynabeads™ (10⁷ beads/mL) with S. enterica (5×10⁶ cells/mL) results in a tight, multi-cell interaction, demonstrating the effectiveness of the anti-Salmonella coating to bind to S. enterica and support its isolation. The optical absorbance properties of Dynabeads™ anti-Salmonella and S. enterica-bound beads are shown in FIG. 1C. The beads alone have a broad absorption peak at 500 nm with a smaller peak at 250 nm, matching peaks reported in other studies.¹⁷ This absorption is primarily due to the iron oxide core, confirmed through further imaging and analysis discussed in FIG. 5. S. enterica has high absorption in the UVC region (100-280 nm) but decreases steadily with increased wavelength; the high absorption at lower wavelengths is primarily due to nucleic acids in the bacteria.¹⁸ Interestingly, the absorption of Dynabeads™ dampens following conjugation to S. enterica. This damping effect is likely due to increased light scattering from bound bacteria resulting in a lower detected absorption coefficient.^{19,20} These absorbance trends are consistent across batches as shown in FIG. 7. Furthermore, we note conjugation of beads to cells consistently maintains the clustering of Dynabeads™ in both liquid and dry sample preparation as shown in FIGS. 1B and 8. As shown in FIG. 1B a single bacteria can bind to two beads at a time, which strengthens and potentially promotes agglomeration upon conjugation of beads with bacteria, in cases where the number densities of the Dynabeads™ and bacteria in the solution are on the same order of magnitude. Details on sample preparation are included in FIG. 9.

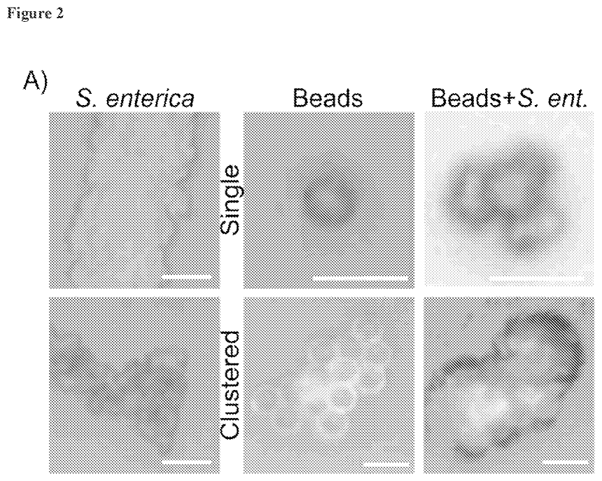

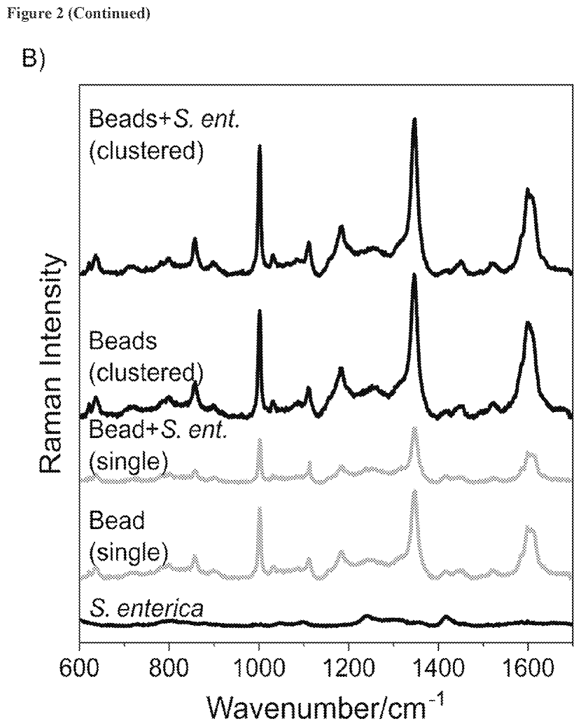

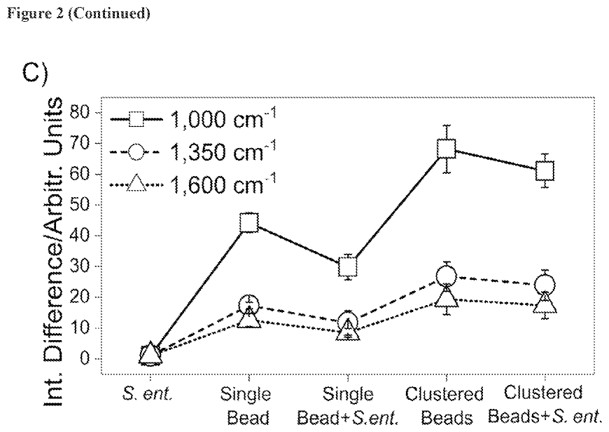

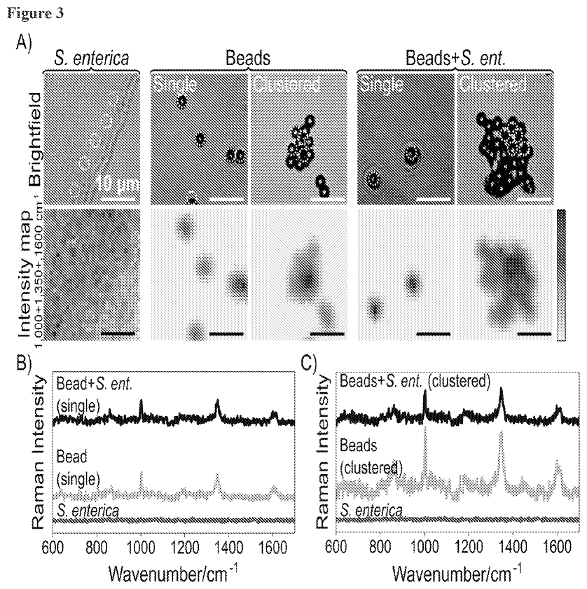

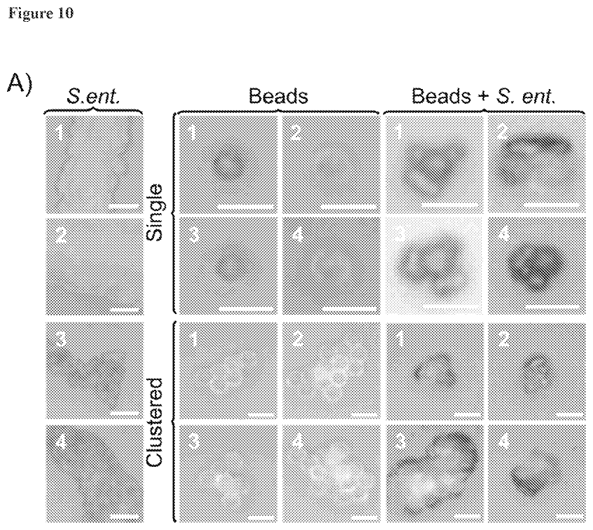

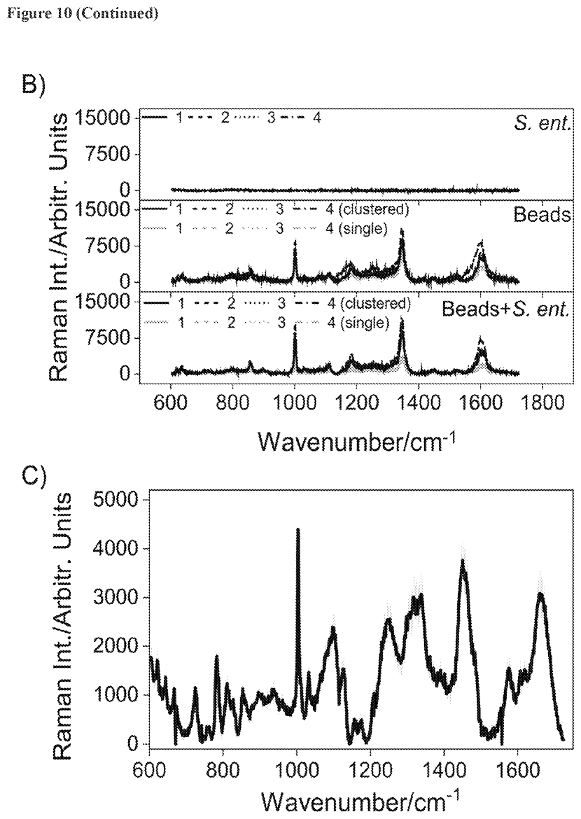

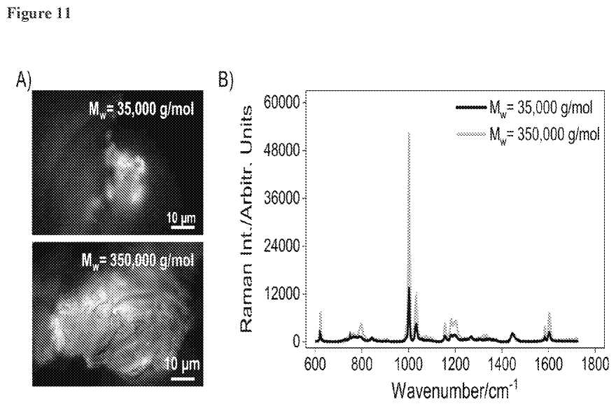

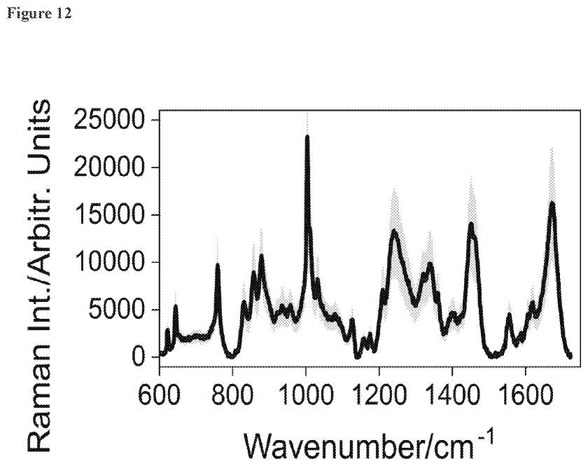

As shown in FIG. 2, Raman spectra of dried samples of single and clustered Dynabeads™ with and without conjugation to S. enterica show a unique signature from a single, 30 sec acquisition at 10 mW laser power. Brightfield images in FIG. 2A show the spots from which measurements in FIG. 2B were collected. As expected, at this acquisition condition S. enterica alone has a weak signal with low intensity, making it difficult to identify signature peaks, typically occurring near 1000, 1400 and 1650 cm⁻¹ (FIG. 10). In a strong contrast, spectra from bead-containing samples display clearly defined peaks at high intensities with a unique Raman signature of Dynabeads™ highlighting their Raman reporter property (FIG. 2B). We attribute characteristic peaks from the Dynabeads™ at 1000 and 1600 cm⁻¹ to aliphatic and aromatic C-C stretching vibrations from the polystyrene coatings of the beads (FIG. 11) and 1350 cm⁻¹ and 1600 cm⁻¹ to the vibrations from amide, α-helix and n-sheet structures of the targeting antibody coupled to the surface of the beads (FIG. 12). Of note, these peaks are prominent both in single and clustered samples allowing detection of signals even from individual beads bound to targets. This is particularly important as target bacteria in fluids of interest such as wastewater, blood and similar biological fluids tend to be very low in number. Notably, higher intensity signature is observed with clustering of beads, which is likely due to increased polystyrene and antibody content at the spot of optical interrogation, resulting in stronger Raman signals (FIG. 2C). Signal intensity difference is calculated with respect to signal intensity of S. enterica only sample analyzed from four different locations. In the case of single beads, the Raman signal intensity from S. enterica-bound Dynabeads™ seems to be dampened compared to signal from single unbound Dynabeads™ (FIG. 2C) with an average of 1.5× larger intensity reading at the three major peak locations 1000, 1350 and 1600 cm⁻¹. This effect could be due to multiple bacteria covering the surface of the polystyrene and antibody coating, scattering the light reducing its optical accessibility for Raman interrogation, an issue that could be amplified in dried sample preparation due to additional refractive index contrast. Interestingly, in the case of clusters (FIG. 3B), signal intensity is minimally and preferentially affected with an average of 1.1× larger intensity at 1000 and 1350 cm⁻¹, and 1.1× dampening at the 1600 cm⁻¹ peak in signal from S. enterica-bound Dynabeads™ compared with their unbound cluster counterparts. This could potentially be because of the locations of cells, which could be arranged around the outer edge of the cluster, leaving a central bead-only portion optically accessible, hence not affecting the signal intensity as much (FIGS. 2B-C and 10). We also note signal dampening could also be due to bead-to-bead polystyrene and antibody coating density variation. As shown in FIGS. 1 and 5, TEM and SEM images show non-uniform polystyrene coating. We would like to emphasize that even though on average such differences are noted, they are within the error range for the four different locations studied making it statistically insignificant when considering large scale measurements in practical applications.

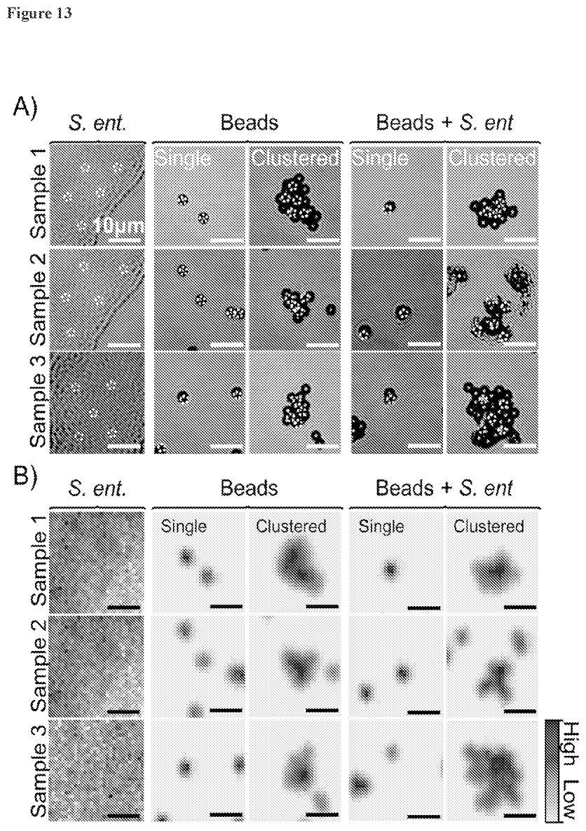

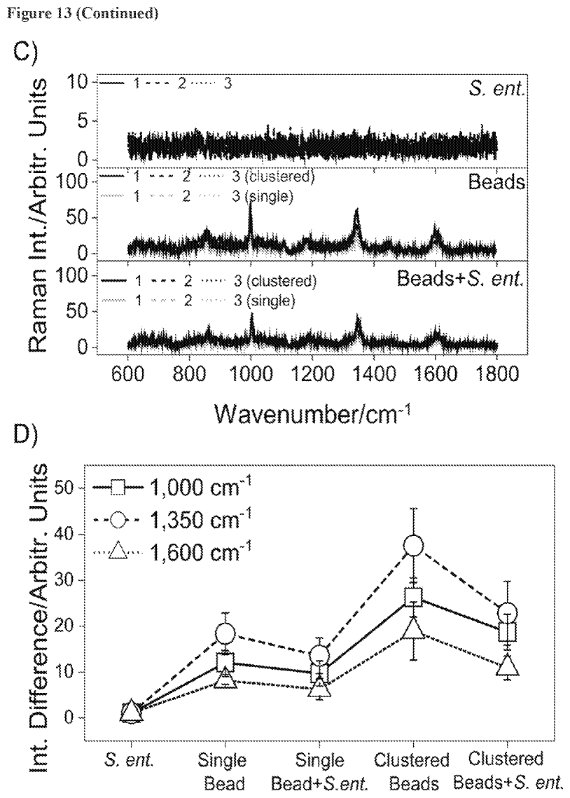

While strong, unique signatures in FIG. 2 are achieved with a 30 s point acquisition using a 10 mW 785 nm laser intensity, these acquisition conditions may be time consuming for large scale applications. Thus, we demonstrate the high throughput target identification potential of Dynabeads™ Raman reporters by capturing spectra from large area of ~30×30 μm using only single shot 0.5 s acquisition with 7 mW laser power as shown in FIG. 3 and FIG. 13.

Brightfield images and intensity maps show the intensity of the Raman shift coming from the site of Dynabeads™, with maximum intensity observed at the center of bead clusters (FIG. 3A). As shown, characteristic peaks from the beads are still identifiable (FIGS. 3B-C) even with markedly reduced detection times, demonstrating the potential for Dynabeads™ Raman reporters in high-throughput single-shot wide-field Raman imaging systems. Bacteria alone have high signal count on the intensity map but have mainly background signal (intensity maps shown are prior to background subtraction) and show no distinct signature peaks at this acquisition time and laser power. The signal intensity from single Dynabeads™ in bound or unbound states is comparable whereas in the case of clusters some dampening is noted upon conjugation as described above.

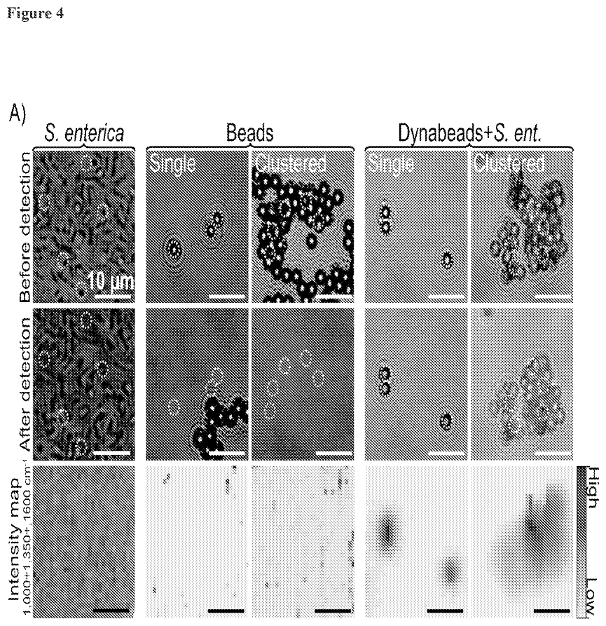

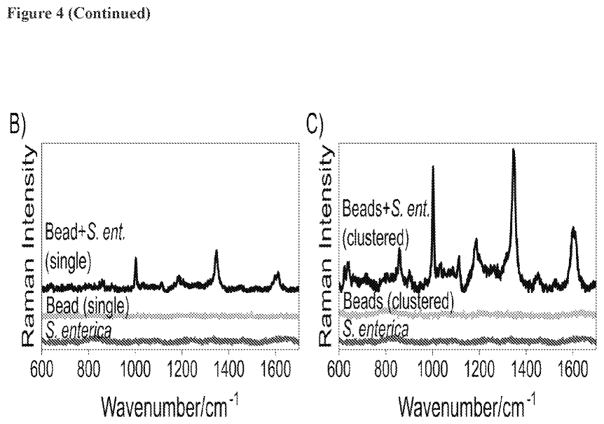

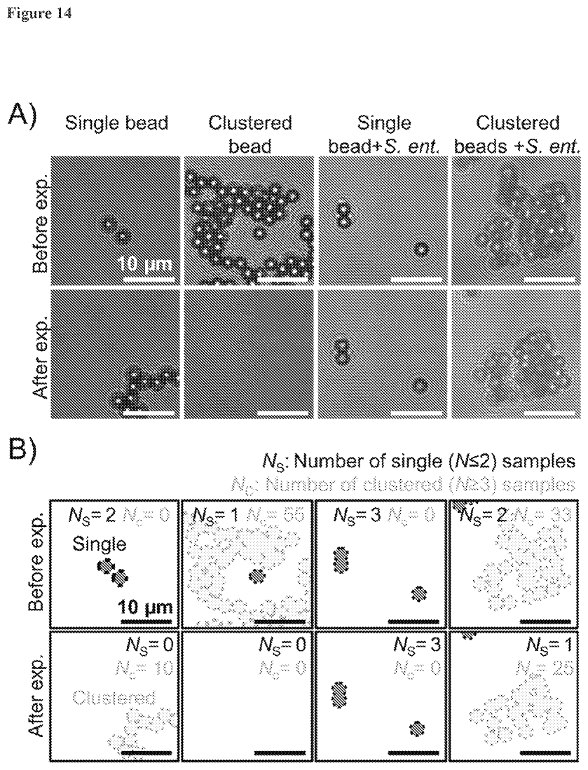

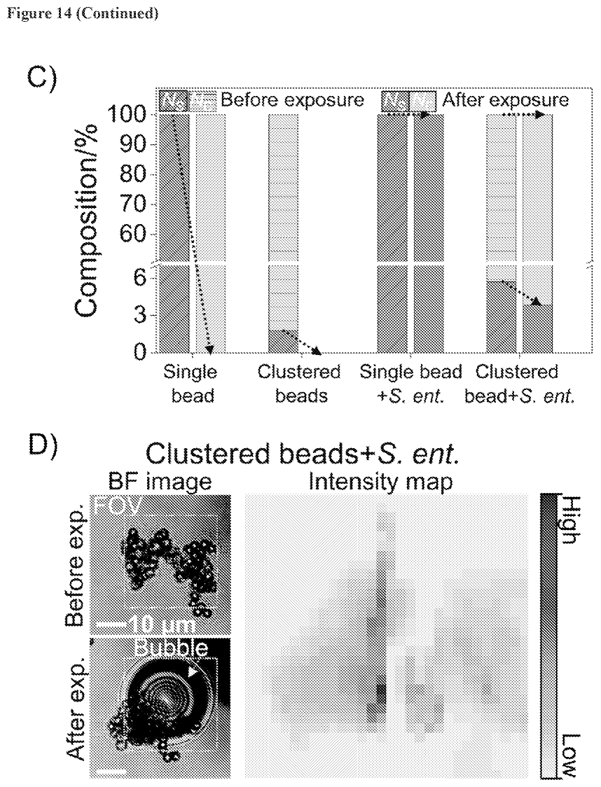

We also demonstrate the application of Dynabeads™ in liquid format, which will enable both simultaneous isolation and detection in complex fluid samples directly from the source. As shown in FIG. 4, the Dynabeads™, Raman reporter signature is preserved in liquid samples. Here, we interrogated solutions of bacteria and Dynabeads™ in DIW after magnetically concentrating S. enterica-bound Dynabeads™ to the bottom of the liquid well, which can be adopted as a scheme for identification of our target foodborne pathogen S. enterica from post-wash wastewater of fruits and vegetables. Interestingly in liquid measurements, brightfield images before and after laser exposure show dislocation S. enterica only and unbound Dynabeads™-only samples upon laser exposure, resulting in small to undetectable signal from bead only samples despite their high intensity signals in dried samples (FIGS. 4A, 14, 15). This is due to unbound cells and beads freely moving through the liquid and dislocating after magnetic concentration and upon laser exposure due to heat-induced convective flow. Notably, however, both single and clustered S. enterica-bound Dynabeads™ remain stable post magnetic concentration and laser exposure and show strong reporter signature consistent with that of Dynabeads™ alone (FIGS. 48-C, 14, and 15). Though aggregation occurs even in Dynabeads™ alone, we believe conjugation to S. enterica results in strongly bound higher mass clumps compared to the Van der Waals force-based clustering of unbound Dynabeads™ as single bacteria can bind to at least two beads at a time. Raman spectra from the selected regions in S. enterica bound beads (dashed circles in FIG. 4A) show similar patterns to those obtained from dried samples (FIGS. 2 and 3) with signature peaks near the wavenumbers of 1000, 1350, and 1600 cm⁻¹. As an additional advantage, in liquid samples, laser powers can be increased 10× that of dried format (75 mW vs 7 mW at 0.5 s acquisition) without melting of the Dynabeads™ as the water serves to dissipate the laser induced thermal effects. These results further emphasize that, through detection of Raman signal from Dynabeads™, the rapid, dual isolation and detection of target bacteria directly from liquid samples positive for bacteria contamination is possible and intrinsically leads to aggregates that concentrate to the imaging surface when an external magnetic field source is applied. We believe this assay is further enabled by and is complementary to emerging methods of separating bound and unbound beads¹⁰ where the Raman signature from the beads serves as a rapid inline detection method even in cases where there are few bacteria in large volumes of solution that are bound by a single Dynabeads™, typical of target samples and routinely requiring culturing and staining steps after separation.

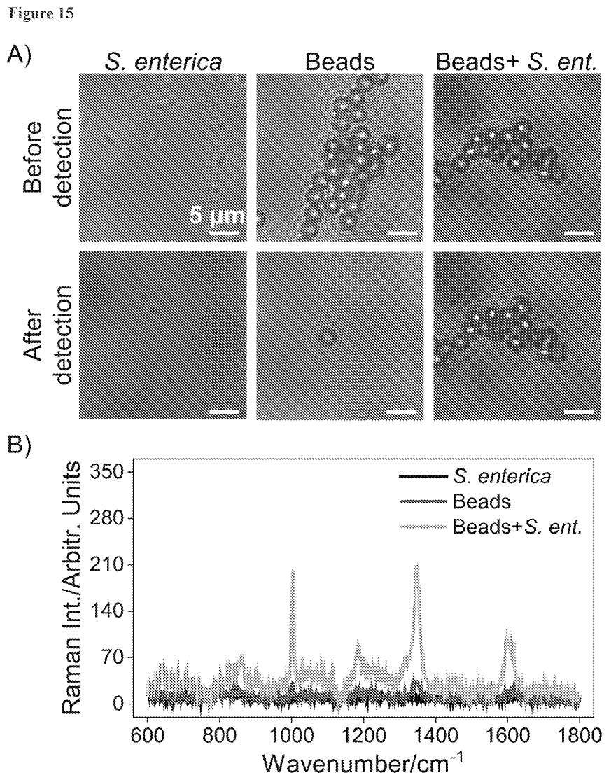

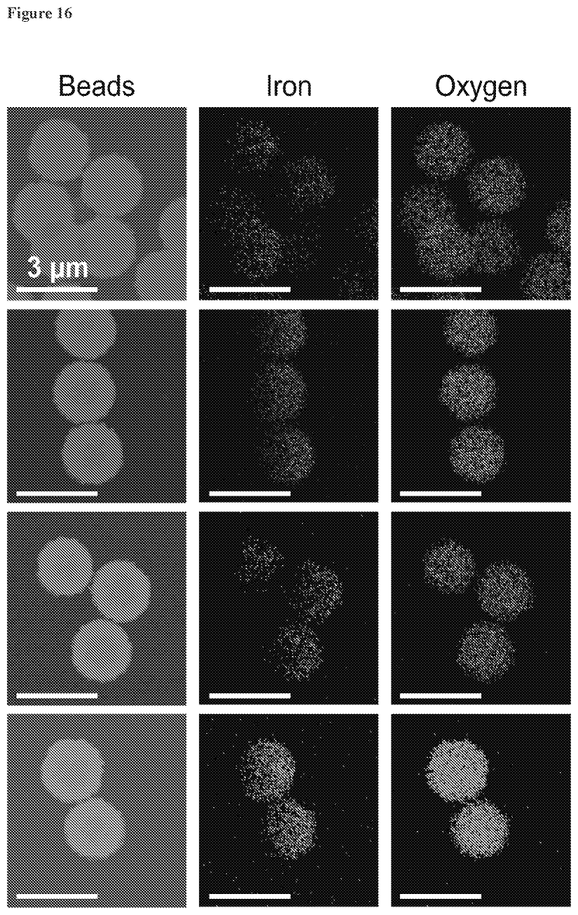

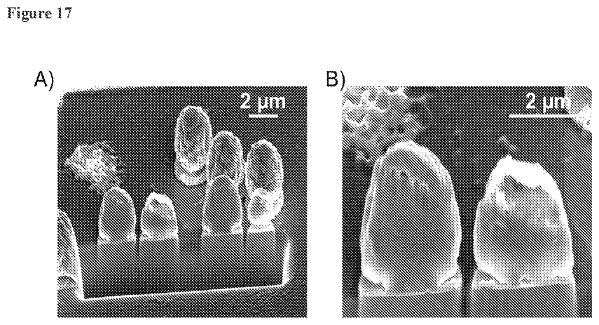

We further characterize the origins of the Raman signature using surface morphology and material composition studies of the Dynabeads™ using SEM, TEM, and energy-dispersive X-ray (EDX) microanalysis as shown in FIG. 5. As expected, Dynabeads™ have a relatively uniform size distribution and an uneven, porous surface morphology consisting of a polymer coating with a maximum thickness of ~150 nm and a−2.44 μm iron oxide core (FIG. 5A) confirmed by EDX spectrum in FIG. 5B. A major carbon peak and minor copper and sulfur peaks are observed which are from the polymer coating, TEM grid and antibody coating respectively. As shown in the inset of FIG. 5B, iron and oxygen are dispersed throughout the core of the bead. This is corroborated by prior studies, where Dynabeads™ were found to be composed of Fe₂O₃ nanoparticles with sizes ranging from 6-12 nm that occasionally form 20 nm clusters.²¹ FIB imaging shown in FIG. 17 did not show a distinct border between the inner core and the outer shell. Interestingly, while oxygen is uniformly distributed, iron shows a single sided preferential distribution consistently across all EDX analysis (see FIG. 16). This could possibly be due to part of the carbon-rich polystyrene coating some iron oxide core nanoparticles. A similar observation was reported in dextran-coated iron oxide nanoparticles.²²

In summary, we have demonstrated the strong Raman reporter activity of polymer-coated magnetic beads, exemplified by commercially available, versatile antibody-coated Dynabeads™ for dual isolation and detection of targets via the recording of Dynabeads™, Raman signature spectra. We note prominent spectral peaks originate from the polystyrene and antibody coating of the Dynabeads™. We show that these beads can be deployed for the simultaneous capture and detection of biomolecules in liquid samples, dried samples and with rapid, single shot high throughput detection schemes. Of note, aggregation of S. enterica-bound Dynabeads™ dramatically increases Raman signal intensities compared to single bound beads due to higher polystyrene and antibody content at the location of measurement, but this effect can be dampened specially in dried samples due to bacteria covering the beads with the additional refractive index contrast and scattering effect limiting access to the polystyrene surface for optical interrogation. In addition, we have illustrated the use of Dynabeads™, superparamagnetic property to magnetically concentrate S. enterica-bound Dynabeads™ to the imaging surface, resulting in stronger Raman signals in liquid samples. The theoretically unlimited target options and available customizations, besides the anti-Salmonella Dynabeads™ discussed here, expands capture and detection applications to diverse cell types and biomolecules. Particularly with advances in separating target-bound Dynabeads™ from unbound Dynabeads™, our demonstration opens a way for rapid interrogation of target-bound beads without additional culturing or staining steps. We believe our Raman based detection approach sheds light on the unique Raman reporter property of versatile Dynabeads™ overcoming current limitations in post-capture target detection, without relying on unique plasmonic substrate engineering lending itself to a wide variety of applications and workflows.

### Methods

S. enterica Preparation and Culture

Salmonella enterica (S. enterica, BAA-710™, ATCC, USA) was grown overnight in tryptic soy broth (TSB) purchased from Sigma-Aldrich (43592). Morning culture was done by reculturing 20 μl of overnight culture in 3 mL of fresh TSB for 3 hrs. The cells were incubated at 37° C., shaken at 400 RPM with 0.1% CO₂. Subsequently, the cells were washed using phosphate buffer saline (PBS, 21-040, Corning®) via centrifugation for 10 mins at 3000 relative centrifugal field (RCF). Quantification of the cells was done using a disposable hemocytometer (inCYTO C-Chip™, DHC-S02, SKC Inc., Korea).

Conjugation/Binding of Anti-Salmonella Dynabeads™ with S. enterica

Anti-Salmonella Dynabeads™ (71002, ThermoFisher Scientific™, Inc.) and S. enterica with concentration ratio of 1:0.5 were incubated in bovine serum albumin (BSA, A3294, Sigma-Adrich@)-blocked 2 mL Eppendorf tubes. The total volume of the reaction was 1 mL in a mixture of PBS with 0.05% Tween® 20 (PBST, P1379, Sigma-Adrich®). The reactants were first vortexed for 1 min then swirled at ambient temperature for 20 min.

### Sample Preparation for Raman Collection

For Dynabeads™—containing samples (Dynabeads™ alone and S. enterica-bound Dynabeads™), a magnet was placed near the sample-containing tube, forming a sample pellet on the tube wall. The buffer was pipetted out and replaced with fresh DIW. For S. enterica, the cell-containing tube was centrifuged for 10 min at 3,000 RCF and the supernatant buffer was replaced with DIW. As shown in FIG. 9, each sample was washed twice with DIW to remove excess buffer for Raman spectral interrogation. To obtain Raman measurements in liquid, 500 μL of liquid from each sample was injected into the silicone isolator (hole with 13 mm-diameter and 2.5 mm-depth, 665307, Grace Bio-Labs, USA)-attached quartz coverslip (25.4×25.4×0.2 mm³, 1×1×0.2, Technical Glass Products, Inc., USA), and a magnet was placed underneath the well for 10 min to pull cell-bound Dynabeads™ down to the quartz coverslip. The magnet was then removed, and the well was placed on the Raman spectroscopy system for spectra collection. For the interrogation of dried samples, 2.5 μL droplets of each sample were drop casted on a quartz coverslip. Nitrogen gas with a pressure of 10 kPa was then blown for 10 min above the deposited droplets to accelerate drying. The samples were then placed on the Raman spectroscopy system for spectra collection. We confirm that the chemicals in the original stock buffers for cells and Dynabeads™-TSB for S. enterica and PBS, BSA, and sodium azide (NaN₃, S2002, Sigma-Adrich®) for Dynabeads™—do not have signature peaks in their Raman spectra, as shown in FIG. 17.

### Raman Spectra Collection

We utilized two Raman spectroscopy systems for collecting Raman spectra from samples with different detection modes: single point detection and mapping mode. For single point detection, the InVia™ Reflex Raman system (Renishaw plc., UK) was utilized to detect Raman spectra from a 1 μm-sized exposing laser spot on the dried sample (i.e., static detection). Single, 30 sec acquisitions at 10 mW power were used to measure spectra from dried samples in FIG. 2. A customized inverted Raman system was utilized to detect Raman spectra from both high-throughput dried and liquid samples and visualize their intensities with single, 0.5 sec acquisitions at 7 or 75 mW power from a field of view (FOV) of 30×30 μm² (i.e., mapping).²³ Each FOV was divided into 900 pixels (30×30 pixels, 1×1 μm²/pixel). We detail the experimental conditions for each Raman spectroscopy measurement in Table 1. We applied the magnet prior to Raman interrogation. But the exact methodology for magnetic concentration is adjustable to fit other applications and Raman systems including sustained magnetic field application. After collecting Raman spectra data, we performed polynomial fitting-assisted background-subtraction using Lieberfit.²⁴ For drawing intensity maps, we picked wavenumbers corresponding to the signature peaks of Dynabeads™; 1000, 1350, and 1600 cm¹, and combined their intensities for clear differentiation of samples in the intensity map. All data, including Raman spectra from single point detection and mapping, were plotted using OriginPro® software (OriginPro® 2023, v10.0.0.154, OriginLab® Corp., USA).

### TEM & EDX Analysis

For imaging under TEM, 10 μL of sample and buffer-containing solution was dropped on a 200-mesh copper grid (Electron Microscopy Sciences, USA) coated with a continuous carbon film and dried at room temperature. The grid was mounted on a JEOL single tilt holder equipped in the TEM column. Imaging on a JEOL 2100 FEG microscope was done using the largest area size of parallel illumination beam and a condenser aperture 100 μm in diameter. The microscope was operated at 200 kV with magnification in the ranges of 3,000 to 600,000 for assessing particle shape, size, and atomic arrangement. All images were recorded on a Gatan Side mounted camera. STEM imaging was done by a HAADF (high-angle annular dark field) detector with 0.5 nm probe size and 12 cm camera length. X-Max 80 mm² EDX (Oxford Instrument, UK) was used for chemical information mapping of samples.

### SEM & FIB Imaging

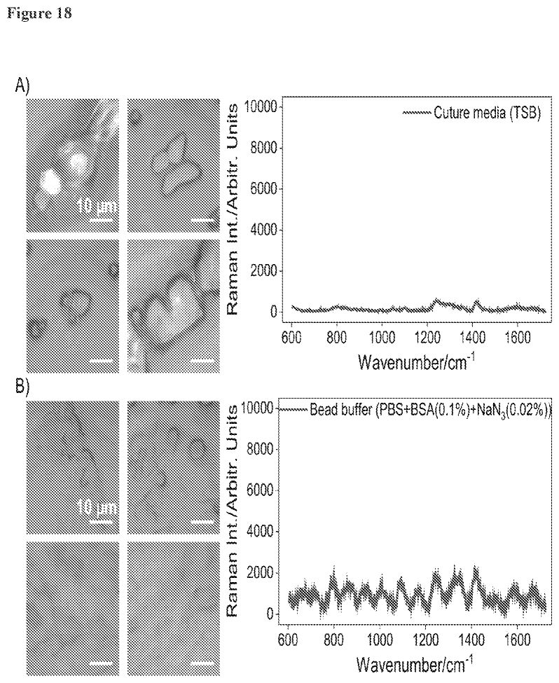

To prepare samples for SEM and FIB analysis, silicon wafers were cut and cleaned with isopropyl alcohol and acetone before mounting on a stub. The stub surface was then sputtered with a 30 nm gold coating. 20 μL of Dynabeads™ anti-Salmonella suspended in DIW at a concentration of 10⁷ beads/mL were drop casted onto the wafer and dried with nitrogen gas at a pressure of 10 kPa before an additional layer was added. Following drying of both layers, the sample area was coated with another 10 nm of gold to reduce charging effects under the electron beam. The beads were then imaged at a working distance of 4.0 mm and magnification ranging from 4989-35000 with the FEI Helios NanoLab™ 600 Dual Beam System at 5 kV and a current of 86 pA. The horizontal field width ranged from 3.66-25.7 μm. 7 μm-deep cross-sectional cuts were made with FIB analysis at 30 kV and a current of 0.46 nA. SEM images were then taken at a stage tilt of 520 and 8012 magnification with a horizontal field width of 16 μm; these images are shown in FIG. 18.

Fluorescence Microscopy of Anti-Salmonella Dynabeads™ and S. enterica

Each 100 μL sample of cells alone, beads alone, or S. enterica-bound beads in PBS buffer was stained with 1 μL of SYTO™ 9 (S34854, ThermoFisher Scientific™ Inc.) solution in DMSO (C=5 mM) at 4° C. overnight and observed using a CCD camera (Andor iXon, Oxford Instruments, UK)-equipped fluorescence microscope (Nikon Eclipse TE200U, Nikon Instruments, NY) after buffer change with fresh PBS.

### UV-Vis Spectroscopy

For UV-Vis analysis, S. enterica-bound Dynabeads™ were washed by magnetization using PBST to remove unbound S. enterica. An Agilent technologies Cary 60 UV-Vis was used to study the optical properties of the S. enterica-bound Dynabeads™, anti-Salmonella Dynabeads™ alone, and S. enterica alone.

It should be understood that the subject matter defined in the appended claims is not necessarily limited to the specific implementations described above. The specific implementations described above are disclosed as examples only.

TABLE 1
Experimental conditions of Raman spectroscopy systems used.

| | | | Laser configuration | | | | |
|---|---|---|---|---|---|---|---|
| | Detection | Sample | Wavelength | Spot size | Power | Exposure | Accumu- |
| System | method | condition | (nm) | (μm) | (mW) | Time (s) | lation |
| Renisha | Single point | Dried | 785 | 1 | 10 | 1 | 30 |
| w Invia | | | | | | | |
| Reflex | | | | | | | |
| Custom- | Imaging mode | Dried | 785 | 1 | 7 | 0.5 | 1 |
| ized | (30 × 30 μm) | Liquid | 785 | 1 | 75 | 0.5 | 1 |

### BIBLIOGRAPHY

1. Ugelstad, J., Söderberg, L., Berge, A. & Bergström, J. Monodisperse polymer particles—a step forward for chromatography. Nature Publishing Group UK http://dx.doi.org/10.1038/303095a0 (1983) doi:10.1038/303095a0.
2. Ugelstad, J., Ellingsen, T., Berge, A. & Helgee, O. B. Magnetic polymer particles and process for the preparation thereof. US Patent (1987).
3. Neurauter, A. A. et al. Cell Isolation and Expansion Using Dynabeads®. in Cell Separation: Fundamentals, Analytical and Preparative Methods (eds. Kumar, A., Galaev, I. Y. & Mattiasson, B.) 41-73 (Springer Berlin Heidelberg, 2007).
4. Cudjoe, K. S. IMMUNOMAGNETIC PARTICLE-BASED TECHNIQUES: OVERVIEW. in Encyclopedia of Food Microbiology (ed. Robinson, R. K.) 1088-1095 (Elsevier, 1999).
5. López-Muñoz, E. & Méndez-Montes, M. Chapter Six—Markers of Circulating Breast Cancer Cells. in Advances in Clinical Chemistry (ed. Makowski, G. S.) vol. 61 175-224 (Elsevier, 2013).
6. Hu, P., Zhang, W., Xin, H. & Deng, G. Single Cell Isolation and Analysis. Front Cell Dev Biol 4, 116 (2016).
7. Awang, M. S. et al. Advancement in Salmonella Detection Methods: From Conventional to Electrochemical-Based Sensing Detection. Biosensors 11, (2021).
8. Liu, H.-B., Du, X.-J., Zang, Y.-X., Li, P. & Wang, S. SERS-Based Lateral Flow Strip Biosensor for Simultaneous Detection of Listeria monocytogenes and Salmonella enterica Serotype Enteritidis. J. Agric. Food Chem. 65, 10290-10299 (2017).
9. Zhou, X. et al. Bacteria Detection: From Powerful SERS to Its Advanced Compatible Techniques. Adv. Sci. 7, 2001739 (2020).
10. Strawser, M. C. Density-shift Immunomagnetic Separation for Pathogen Retrieval from Complex Media. (Massachusetts Institute of Technology, 2022).
11. Tadesse, L. F. et al. Toward rapid infectious disease diagnosis with advances in surface-enhanced Raman spectroscopy. J. Chem. Phys. 152, 240902 (2020).
12. Zhang, L. et al. Multifunctional magnetic-plasmonic nanoparticles for fast concentration and sensitive detection of bacteria using SERS. Biosensors and Bioelectronics 31, 130-136 (2012).
13. Jun, B.-H. et al. Protein separation and identification using magnetic beads encoded with surface-enhanced Raman spectroscopy. Anal. Biochem. 391, 24-30 (2009).
14. Cha, H. et al. Surface-enhanced Raman scattering-based immunoassay for severe acute respiratory syndrome coronavirus 2. Biosens. Bioelectron. 202, 114008 (2022).
15. Gonzalez, S. V., Nair, M. N., Belk, K. E. & Geomaras, I. Efficacy of Antimicrobial Spray Treatments in Reducing Salmonella enterica Populations on Chilled Pork. J. Food Prot. 86, 100068 (2023).
16. Bai, L. et al. Rapid, Visual, and Sequence-Specific Detection of Salmonella in Egg Liquid with vis-NEAA, a CRISPR/Cas12 Empowered New Strategy. J. Agric. Food Chem. 70, 2401-2409 (2022).
17. Guo, L. et al. Sensitive detection of cardiac troponin T based on superparamagnetic bead-labels using a flexible micro-fluxgate sensor. RSC Adv. 7, 52327-52336 (2017).
18. Dai, T., Vrahas, M. S., Murray. C. K. & Hamblin, M. R. Ultraviolet C irradiation: an alternative antimicrobial approach to localized infections?Expert Rev. Anti. Infect. Ther. 10, 185-195 (2012).
19. Waltham, C., Boyle, J., Ramey, B. & Smit, J. Light scattering and absorption caused by bacterial activity in water. Appl. Opt. 33, 7536-7540 (1994).
20. Klein, R., Braun, W., Fahr, A., Mele, A. & Okabe, H. Scattered Light and Other Corrections in Absorption Coefficient Measurements in the Vacuum Ultraviolet: A Systems Approach. J. Res. Natl. Inst. Stand. Technol. 95, 337-344 (1990).
21. Fonnum, G., Johansson, C., Molteberg, A., Morup, S. & Aksnes, E. Characterisation of Dynabeads@ by magnetization measurements and Møssbauer spectroscopy. J. Magn. Magn. Mater. 293, 41-47 (2005).
22. Predescu, A. M. et al. Synthesis and characterization of dextran-coated iron oxide nanoparticles. R Soc Open Sci 5, 171525 (2018).
23. Kang, J. W., So, P. T. C., Dasari, R. R. & Lim, D.-K. High resolution live cell Raman imaging using subcellular organelle-targeting SERS-sensitive gold nanoparticles with highly narrow intra-nanogap. Nano Lett. 15, 1766-1772 (2015).
24. Lieber, C. A. & Mahadevan-Jansen. A. Automated method for subtraction of fluorescence from biological Raman spectra. Appl. Spectrosc. 57, 1363-1367 (2003).
25. Wen, Z.-Q. Raman spectroscopy of protein pharmaceuticals. J. Pharm. Sci. 96, 2861-2878 (2007)

1. A method of detection, wherein the method comprises
(a) obtaining a sample, wherein the sample comprises target selected from biological cells and viruses;
(b) incubating the sample with polymer-coated magnetic beads that comprise an affinity agent for a target of interest on a bead surface, to produce bead-target complexes;
(c) analyzing the bead-target complexes by Raman spectroscopy to produce a spectrograph; and
(d) detecting the presence of the bead-target complexes by analyzing the spectrograph.

2. The method of claim 1, wherein the polymer-coated magnetic beads comprise polymer-coated superparamagnetic beads.

3. The method of claim 1, wherein the polymer-coated magnetic beads comprise polystyrene-coated magnetic beads.

4. The method of claim 3, wherein the analyzing the bead-target complex by Raman spectroscopy measures polystyrene of the polystyrene-coated magnetic bead.

5. The method of claim 3, wherein the analyzing the bead-target complex by Raman spectroscopy measures Raman intensity at 700 to 1700 cm¹.

6. The method of claim 1, a wherein the affinity agent comprises an antibody.

7. The method of claim 1, wherein the target comprises biological cells.

8. The method of claim 1, wherein the bead-target complex has a different density than the polymer-coated magnetic bead.

9. The method of claim 1, further comprising separating the bead-target complex from the polymer-coated magnetic bead.

10. The method of claim 9, wherein a density gradient is used in separating the bead-target complex from the polymer-coated magnetic bead.

11. The method of claim 10, wherein the density gradient comprises two layers, wherein the density of the two layers differs.

12. The method of claim 10, wherein the density gradient comprises a continuous density gradient.

13. The method of claim 1, wherein the analyzing the bead-target complex by Raman spectroscopy measures only the bead-target complex and not polymer-coated magnetic beads free of target.

14. The method of claim 9, wherein the separating the bead-target complex is performed based on the density of the bead-target complex.

15. The method of claim 9, wherein the separating the bead-target complex is performed based on the magnetic properties of the polymer-coated magnetic bead.

16. A method of enriching, the method comprising
a. obtaining a sample, wherein the sample comprises target selected from biological cells and viruses;
b. incubating the sample with polymer-coated magnetic beads that comprise an affinity agent for target of interest on a bead surface, to produce bead-target complexes; and
c. separating the bead-target complexes from the polymer-coated magnetic beads based on the density of the bead-target complex;
wherein the separating the bead-target complex enriches a relative concentration of the bead-target complexes.

17. The method of claim 16, wherein the polymer-coated magnetic beads comprise polymer-coated superparamagnetic beads.

18. The method of claim 16, further comprising separating the bead-target complex based on the magnetic properties of the polymer-coated magnetic beads.

19. The method of claim 16, wherein affinity agent comprises an antibody.
